(19) United States
(12) Patent Application Publication
(10) Pub. No.: US 2015/0151077 A1
(43) Pub. Date: Jun. 4, 2015

(54) Devices And Methods For Renal Denervation

(71) Applicant: Douglas C. HARRINGTON, Los Altos Hills, CA (US)

(72) Inventor: Douglas C. Harrington, Los Altos Hills, CA (US)

(21) Appl. No.: 14/407,458

(22) PCT Filed: Jun. 13, 2013
(86) PCT No.: PCT/US13/45715

§ 371 Date: Dec. 11, 2014

Related U.S. Application Data
(60) Provisional application No. 61/659,343, filed on Jun. 13, 2012.

(51) Int. Cl. A61M 25/00; A61L 29/16; A61M 31/00

(52) U.S. Cl. CPC A61M 25/0084; A61M 31/007; A61M 25/003; A61M 25/0074; A61L 29/16; A61M 2210/1082; A61M 2025/0037; A61M 2025/0086

(57) ABSTRACT

Devices and methods that produce alterations of renal sympathetic nerve activity by use of tissue modifying implants. Devices for percutaneous delivery of implants into a renal artery or vein wall employing various needle assembly arrangements to modify renal nerve activity.

### RELATED APPLICATIONS

This application claims priority to U.S. Provisional Application Ser. No. 61/659,343 filed Jun. 13, 2012 entitled Devices And Methods For Renal Denervation, which is hereby incorporated herein by reference in its entirety.

### BACKGROUND OF THE INVENTION

Hypertension or abnormally high blood pressure is a growing public health concern for which successful treatment often remains elusive. In the United States, about 50 million people age six and older have high blood pressure.

Hypertension is more common in men than women and afflicts approximately 50% of the population over the age of 65. Hypertension is serious because people with the condition have a higher risk for heart disease and other medical problems than people with normal blood pressure. If left untreated, hypertension can lead to arteriosclerosis, heart attack, stroke, enlarged heart and kidney damage.

Blood pressure is highest when the heart beats to push blood out into the arteries. When the heart relaxes to fill with blood again, the pressure is at its lowest point. Blood pressure when the heart beats is called systolic pressure. Blood pressure when the heart is at rest is called diastolic pressure. When blood pressure is measured, the systolic pressure is stated first and the diastolic pressure second. Blood pressure is measured in millimeters of mercury (mm Hg). For example, if a person's systolic pressure is 120 and diastolic pressure is 80, it is written as 120/80 mm Hg. Blood pressure lower than 120/80 mm Hg is considered normal.

A significant percentage of patients with uncontrolled hypertension fail to meet therapeutic targets despite taking multiple drug therapies at the highest tolerated doses, a phenomenon called resistant hypertension. This suggests there is an underlying pathophysiology resistant to current pharmacological approaches. Innovative therapeutic approaches are particularly relevant for these patients, as their condition puts them at high risk of major cardiovascular events.

The sympathetic nerve innervation of the kidney is implicated in the pathogenesis of hypertension through effects on rennin secretion, increased plasma rennin activity that leads to sodium and water retention, and reduction of renal (kidney) blood flow. As a result, a succession of therapeutic approaches has targeted the sympathetic nervous system to modulate hypertension, with varying success.

The sympathetic nerve innervation of the kidney is achieved through a dense network of postganglionic neurons that innervate the kidney. The axon of preganglionic neurons exits the thoracic and lumbar sympathetic trunk and reach the pre- and par-avertebralsympathetic ganglia. Renal preganglionic nerves run alongside the renal artery and enter the hilus of the kidney. Thereafter, they divide into smaller nerve bundles following the blood vessels and penetrate cortical and juxtamedullary areas. Renal sympathetic nerve activation enhances noradrenalin production for nerve endings and noradrenalin spillover, while interruption of renal sympathetic fibers results in a marked decrease of noradrenalin spillover. When renal sympathetic nerves are activated, b1 adrenergic receptors enhance rennin secretion and a1 receptor activation results in increase sodium and fluid reabsorption, renal vasoconstriction, and decrease in renal blood flow.

Afferent renal sympathetic nerves originate mostly from the renal pelvic wall. The cell bodies of renal afferent nerves lie in the ipsilateral dorsal root ganglia. From there, ascending signals travel to the renal cardiovascular centers in the central nervous system. Afferent renal nerve activation promotes vasopressin and oxytocin release from the neuro-hypophyisis. Prior renal denervation (interruption of the nerve connections) of the stimulated kidney, however, attenuates these effects, suggesting that complete renal denervation effectively inhibits ascending afferent stimuli. Overall afferent sympathetic fibers may have important contribution in regulation of systemic vascular resistance and blood pressure control.

As a result of the renal afferent and sympathetic efferent nerves being implicated in the pathophysiology of systemic hypertension, a succession of therapeutic approaches have targeted the sympathetic nervous system to modulate hypertension, with varying success.

Surgical sympathectomy, the surgical cutting of a sympathetic nerve or removal of a ganglion, was attempted more than 40 years ago in patients with malignant hypertension. Malignant hypertension was a devastating disease with a five-year mortality rate of almost 100%, thus interventional approaches have been tested for its treatment given the lack of effective drug therapy at the time. Sympathectomy was mainly applied in patients with severe or malignant hypertension, as well as patients with cardiovascular deterioration despite relatively good blood pressure reduction by other means.

Sympathectomy, also termed splanchnicectomy, had to include the abdominal organs in order to be effective. The procedure was performed either in one or two stages, required a prolonged hospital stay (2-4 weeks) and a long recovery period (1-2 months) and importantly had to be performed by a highly skilled surgeon. It was thus performed only in a few select centers in the U.S. and Europe.

Sympathectomy proved to be effective in reducing blood pressure immediately postoperatively, and the results were maintained in the long term in most patients. Survival rates were also demonstrated to be high for patients undergoing the procedure. The two major limitations of splanchnicectomy were the required surgical expertise and the frequent adverse events occurring with this procedure. Adverse events were common and included orthostatic hypotension (very low blood pressure when standing up), orthostatic tachycardia, palpitations, breathlessness, anhidrosis (lack of sweating), cold hands, intestinal disturbances, sexual dysfunction, thoracic duct injuries and atelectasis (collapse of the lung).

After the introduction of antihypertensive drugs and due to its poor patient tolerance and surgical difficulty, sympathectomy was reserved for patients who failed to respond to antihypertensive therapy or could not tolerate it.

Recent research has focused on using thermal energy delivered through a percutaneous approach to achieve renal denervation. Renal denervation performed this way is designed to damage the renal nerves using hot or cold energy to block renal nerve activity, thus neutralize the effect of the renal sympathetic system which is involved in the development of hypertension. Percutaneous thermal device based renal denervation may achieve such objectives, but could also produce possible complications from the delivery of energy including aberrant burns affecting adjacent organs and structures and significant trauma to the inner surface of the vessel leading to stenosis, occlusion and/or embolisms.

Another percutaneous approach discussed involves delivery of a liquid agent directly to the renal nerves to achieve renal denervation. Due to the unpredictable nature of fluid travel within the various tissues, targeting renal nerves with this approach is difficult and may lead to random clinical results. Also of concern with using a liquid agent is the high probability of fluid migration and intravasation which could result in inadvertent adjacent organ and structural damage systemically should the fluid escape back in the vessel lumen.

There is the need for a method and device that can perform renal denervation without the risks associated with thermal energy and liquid agents.

### SUMMARY OF THE INVENTION

The invention relates to devices and methods for treating hypertension and its related conditions. The method involves percutaneous delivery of solid bioactive materials in proximity to renal nerves using a catheter. Delivery of the material causes renal denervation by creating a tissue response that result in a decrease or cessation of renal nerve activity involved in the development of hypertension.

Embodiments of the present invention are directed to a catheter assembly consisting of needle elements located at the distal end of said catheter. Solid implants or pellets are contained within the needle elements. One method involves percutaneous placement of the catheter into the renal artery, advancement of the needle into the vessel wall and deployment of the implants within the vessel wall in proximity to the renal nerves. The implant generates renal denervation by creating a tissue response that disrupts the renal nerves and remodels the local tissue to prevent nerve tissue regeneration. This area of tissue disruption is herein referred to as the “remodeled zone”. Use of a solid bioactive implant in this manner allows for accurate and precise treatment of the renal nerves with minimal damage to the vessel lumen surface and no damage to adjacent organs or structures.

The renal nerves are normally oriented longitudinal within and along the vessel wall. Complete renal denervation is achieved when a full loop of tissue perpendicular to the longitudinal axis is captured in the remodeled zone resulting in a circumferential block of nerve impulses. One embodiment of the present invention creates remodeled zones that span a complete closed loop perpendicular to the longitudinal axis of the vessel. Needle elements of this embodiment are positioned circumferentially within discrete segments of the catheter perpendicular to the longitudinal axis. Another embodiment of the present invention creates remodeled zones that span one or more open arc segments around the longitudinal axis, but the remodeled zones of all the needle elements inserted longitudinally into any lateral plane which is perpendicular to the longitudinal axis span a substantially closed loop around the longitudinal axis. Because the remodeled zones do not form a closed loop, the risk of renal artery stenosis is decreased. Conversely, because the remodeled zones of the needle elements projected longitudinally into the lateral plane span a substantially closed loop, complete renal denervation is achieved.

In accordance with an aspect of the current invention, an implant delivery catheter comprises an elongated catheter body extending longitudinally between a proximal end and a distal end along a longitudinal axis and a needle element assembly comprising one or a plurality of needle elements connected to the catheter body, each element to be utilized to deliver implants to produce a remodeled zone. The needle elements are distributed in a circumferential or angled configuration such that the remodeled zones span one or more open arc segments around the longitudinal axis, and the remodeled zones projected longitudinally into any lateral plane which is perpendicular to the longitudinal axis span a substantially closed loop around the longitudinal axis. In most embodiments of the present invention, the needle element assembly is movable between a collapsed arrangement and an expanded arrangement.

In one embodiment of the present invention there is a plurality of needle elements each containing multiple implants. This embodiment delivers the implants employing multiple insertions of the needle elements and placement of implants in various locations of the vessel so that there are several loops of remodeled zones perpendicular to the longitudinal axis. In another embodiment of the present invention there is a single needle element that contains multiple implants. This embodiment delivers the implants to targeted tissue employing multiple insertions of the needle element and placement of implants in various locations of the vessel, preferably so that the remodeled zone is a complete open or closed loop perpendicular to the longitudinal axis.

In accordance with an aspect of the invention, an implant delivery catheter comprises an elongated catheter body extending longitudinally between a proximal end and a distal end along a longitudinal axis and a hollow coil element assembly connected to the catheter body comprising a plurality of needle elements to be utilized for delivery of implants to produce a remodeled zone. Coil element has a proximal end connected to the catheter body and a distal end. Coil element is movable between a collapsed configuration and an expanded configuration. Needle elements are located within hollow coil element and are projected laterally outward through holes in the walls of the coil into vessel wall for delivery of implants.

In accordance with an aspect of the invention, an implant delivery catheter comprises an elongated catheter body extending longitudinally between a proximal end and a distal end along a longitudinal axis and a balloon element assembly connected to the catheter body comprising a plurality of needle elements to be utilized for delivery of implants to produce a remodeled zone. Balloon element has a proximal end connected to catheter body and a distal end. Balloon element is movable between a collapsed configuration and an expanded configuration. Expanded balloon element shape can be of various configurations including straight and spiral. Tubular elements containing needle elements are attached to the surface of the balloon. Needle elements are projected laterally outward from within tube elements into vessel wall for delivery of implants.

In accordance with an aspect of the invention, an implant delivery catheter comprises an elongated catheter body extending longitudinally between a proximal end and a distal end along a longitudinal axis and a malecot element assembly connected to the catheter body comprising a plurality of needle elements to be utilized for delivery of implants to produce a remodeled zone. Malecot element has a proximal end connected to catheter body and a distal end. Malecot element is movable between a collapsed configuration and an expanded configuration. Expanded malecot element shape can be of various configurations including straight and spiral. Tubular elements containing needle elements are attached to the surface of the malecot. Needle elements are projected laterally outward from tube elements into vessel wall for delivery of implants.

Percutaneous placement of the delivery catheter to the renal artery may be accomplished using any of the currently available techniques and ancillary equipment for renal artery interventions including guided sheaths, steerable distal tip assemblies and over the wire configurations employed for diagnostic and therapeutic devices. There may be other means to place solid implants into the vessel wall not specifically described in one of the inventions embodiments, but it is to be understood that the description is not meant as a limitation since further modifications may suggest themselves or be apparent to those skilled in the art.

The invention disclosed herein may be utilized for treatment of other clinical conditions influenced by renal nerve activity including kidney disease, congestive heart failure, obstructive sleep apnea, diabetes and others. Invention and methods may also be employed to treat clinical conditions unrelated to renal nerve activity, for example by placement of implants around the connections of the pulmonary veins to the left atrium to treat atrial fibrillation.

### BRIEF DESCRIPTION OF THE DRAWINGS

FIG. 1 is an anterior view of human kidneys and supporting vasculature.

FIG. 2 is a posterior view of human kidneys and supporting vasculature.

FIGS. 3a and 3b are sliced cross-sectional and longitudinal views of a human renal artery.

FIGS. 4a-4d are close up distal views of a needle element containing a single implant.

FIGS. 5a-5c are close up distal views of a needle element containing multiple implants.

FIGS. 6a-6f are multiple views of a single straight needle embodiment of the invention.

FIGS. 7a-7f are multiple views of a multiple straight needle embodiment of the invention.

FIGS. 8a-8j are multiple views of the implant placed within the renal artery wall using the multiple straight needle device in FIG. 7.

FIGS. 9a-9f are multiple views of a multiple spiral needle embodiment of the invention.

FIGS. 10a-10h are multiple views of the implants placed within the renal artery wall using the multiple spiral needle device in FIG. 9.

FIGS. 11a-11f are multiple views of a coil needle embodiment of the invention.

FIGS. 12a-12h are multiple views of a balloon needle embodiment of the invention.

FIGS. 13a-13h are multiple views of a spiral balloon needle embodiment of the invention.

FIGS. 14a-14h are multiple views of a malecot needle embodiment of the invention.

### DESCRIPTION OF THE INVENTION

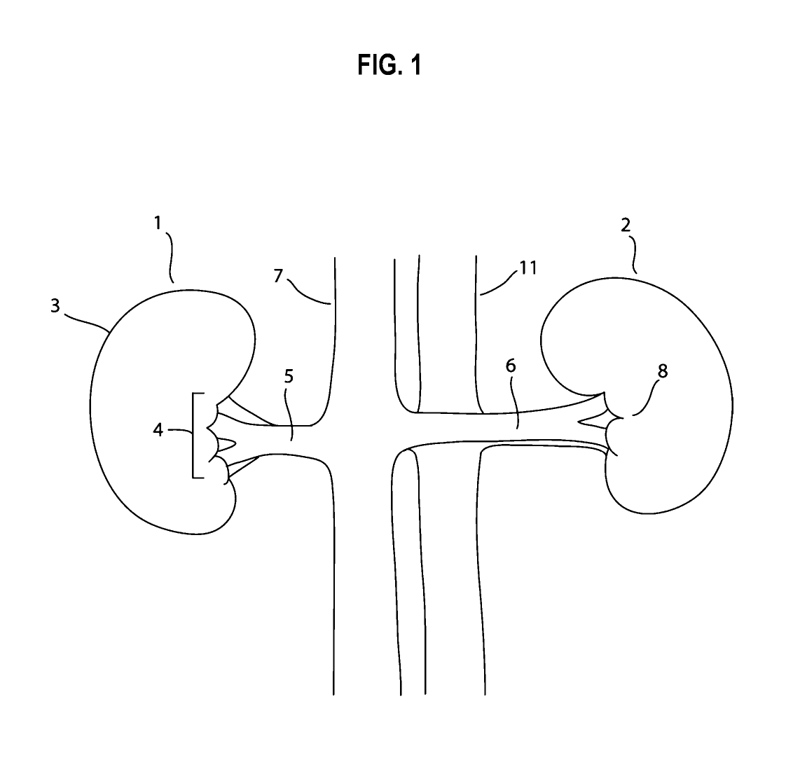

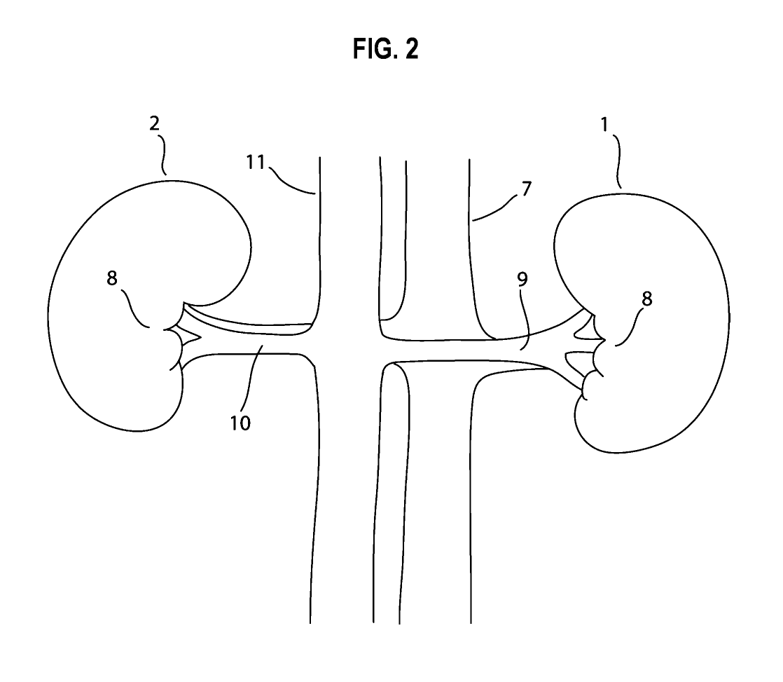

FIG. 1 is an anterior view illustration of the kidneys and major arteries and veins supporting the kidneys. The right kidney 1 and left kidney 2 are bean-shaped organs, each approximately the size of a tightly clenched fist. They lie on the posterior abdominal wall behind the peritoneum and on either side of the vertebral column while the superior pole of each kidney is protected by the rib cage. A fibrous connective tissue renal capsule 3 surrounds each kidney and around the capsule is a dense deposit of adipose tissue, the renal fat pad (not shown), which protects the kidney and supporting vasculature. On the medial side of each kidney is a relatively small area called the helium 4 where the renal artery and the nerves enter and the renal vein and the ureter (not shown) exit. The right renal vein 5 and left renal vein 6 branches off the inferior vena cava 7 and enters the renal sinus 8 of each kidney. Renal veins are blood vessels that carry deoxygenated blood out of the kidney to the inferior vena cava 7. FIG. 2 is a posterior view illustration of the kidneys and major arteries and veins supporting the kidneys. The right renal artery 9 and left renal artery 10 branches off the abdominal aorta 11 and enter the renal sinus 8 of each kidney. The renal arteries carry a large portion of total blood flow to the kidneys. Up to a third of total cardiac output can pass through the renal arteries to be filtered by the kidneys.

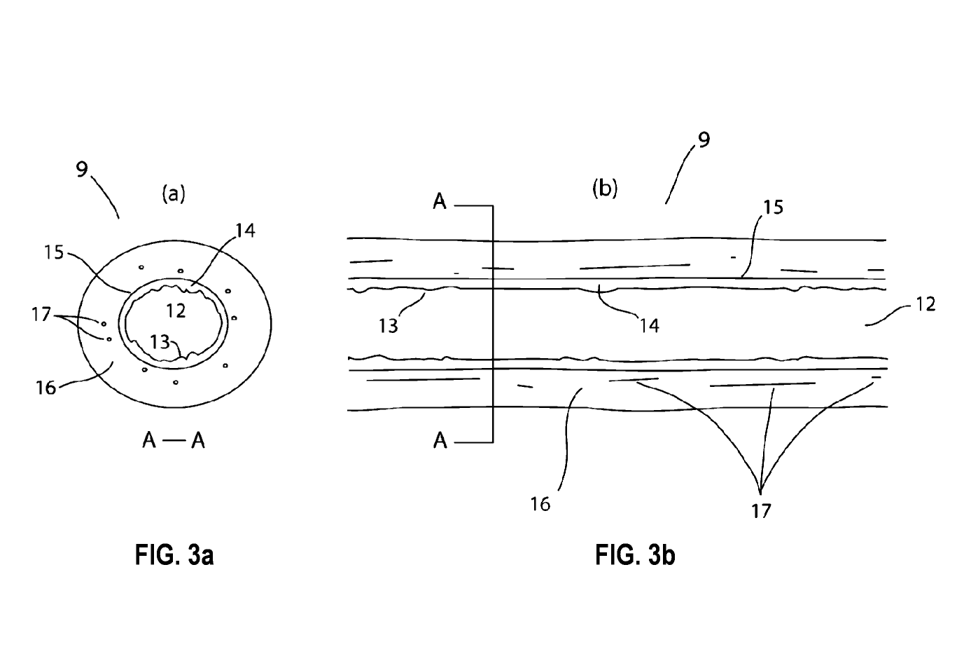

FIG. 3 is an illustration of the renal artery 9 including renal nerves. FIG. 3a is sliced cross-section of the renal artery and FIG. 3b is a sliced longitudinal section of the renal artery showing the vessel lumen 12 and vessel wall layers. The tunica intima 13 or inner vessel lumen surface layer is a thin membrane that mainly consists of endothelium and lamina propria. The tunica media 14 or middle layer consists of smooth muscle tissue, elastic and collagen fibers. At the outer border of the tunica media an external elastic membrane 15 separates the tunica media from the outer layer, the tunica adventitia 16. The tunica adventitia 16 is composed of connective tissue, which varies from dense connective tissue that is near the tunica media 14 and contains large amounts of collagen to loose connective tissue that merges with the connective tissue surrounding the blood vessel. The sympathetic nerve innervation of the kidney is achieved through a dense network of postganglionic neurons that innervate the kidney. Renal nerves 17 located in the tunica adventitia 16 run in a relatively longitudinal direction alongside the renal artery 9 and enter the hilus 4 of the kidney 1.

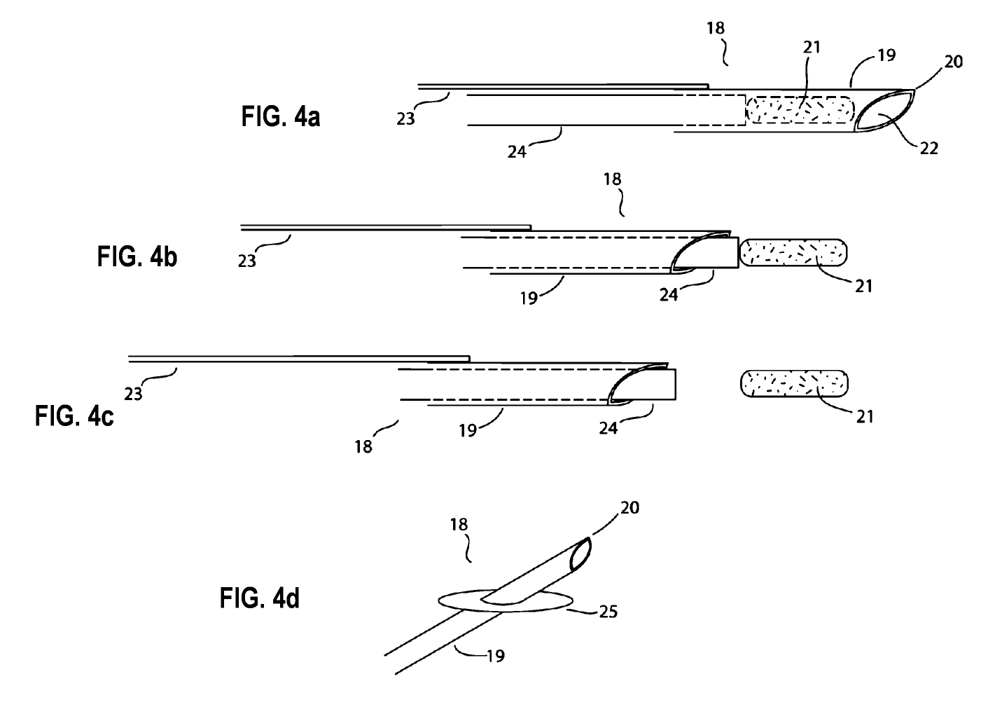

FIG. 4a is a partial longitudinal view of the needle element 18. The hypodermic needle 19 is a typically rigid or semi-ride longitudinal tubular structure with a proximal end leading into the delivery catheter body (not shown) and a sharp pointed distal end 20 to aid with insertion into vessel wall. Implant 21 (shown with phantom lines) is stored within the needle lumen 22. Proximal end of the needle is mechanically attached to a rigid element 23 within the catheter body and extends the length to the proximal end of the catheter body. A holding rod 24 is disposed within the catheter body and extends the length to the proximal end of the catheter body. Hypodermic needle 19 and holding rod 24 can be advanced or retracted by the operator by various means including wires, hand held mechanisms and handles with activation mechanism.

In use, catheter body is positioned within targeted vessel lumen 12 and maintained in a fixed positioned by the operator. Needle element 18 containing implant 21 is advanced from distal end of catheter and pierced and inserted into the vessel wall while both the hypodermic needle 19 and holding rod 24 are mechanically coupled. Once the needle element 18 is in the preferred location in the vessel wall, hypodermic needle 19 and holding rod 24 are decoupled and hypodermic needle 19 withdrawn from vessel wall by pulling the rigid element 23 proximally while holding the catheter body and holding rod 24 in a relatively fixed position (FIG. 4b). This operation ejects the implant 21 without the need for relative motion between the implant 21 and injection site after the operator has positioned the catheter for use. Once hypodermic needle 19 if fully withdrawn, needle element 18 is removed from vessel injection site leaving implant 21 in targeted location within vessel wall (FIG. 4c).

It may be desirable to control the insertion depth of the needles to accurately target the renal nerves and prevent any undesired damage to deeper tissues. Various techniques and mechanisms can be employed to control the insertion depth of the needle into the vessel wall such as adding mechanical stoppers to the hypodermic needle 19. For example, FIG. 4d shows a hypodermic needle 19 with a rigid or semi-rigid circular disk 25 mechanically attached at a point proximal from the distal end of hypodermic needle. In use, as the needle element 18 is advanced into the vessel wall, advancement is arrested once the circular disk 25 engages the vessel wall, thus preventing deep penetration of the needle element 18. Depth of penetration of needle element can be 200 microns to several centimeters depending upon needle geometry, vessel wall insertion angle, and targeted tissue location.

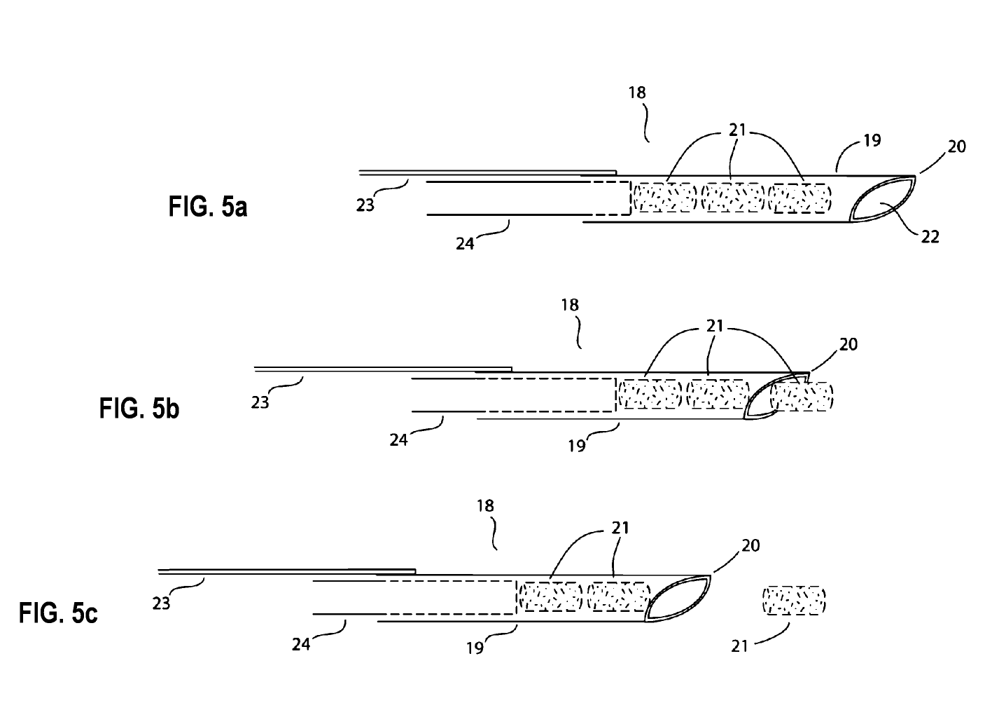

FIG. 5a is a partial longitudinal view of the needle element 18 containing multiple implants. Implants 21 (shown with phantom lines) are stored within the needle lumen 22. Device with this embodiment allows for multiple implants in one injection location or distribution of multiple implants and injection locations of vessel wall using the same needle element 18. In use, hypodermic needle 19 is withdrawn a fixed distance to detach the first implant 21 but continue to house the remaining implants 21 within the needle lumen 22 as shown in FIGS. 5b-5c. Insertion and implant ejection is repeated in different locations of the vessel lumen using the same needle element 18 until treatment is complete or the implants 21 are exhausted. One advantage of this embodiment is the requirement of only one catheter to complete treatment on both right and left renal arteries.

Implant 21 is composed of a solid material, preferably bioactive. After placement into vessel wall, implant creates a tissue response that disrupts and remodels the local tissue containing nerves to suppress or eliminate nerve tissue activity temporarily or permanently. Implants can be either non-degradable (permanent) or biodegradable (e.g. absorbable surgical sutures) which will gradually break down and be absorbed by the body after implantation. Any suitable implant material, both organic and inorganic, as well as combinations thereof may be used. The material of the implant may be solid, braided or woven from a single material or a combination of materials.

One class of material suitable for this application is sclerosants. A sclerosant is an irritant that elicits local tissue inflammation and subsequent fibrosis (scar) to form. Sclerosants are currently employed for treatment of various diseases including varicose veins, hemorrhoids, esophageal varices, pleural effusion and Morton's neuroma. The preferred sclerosing implant would have no systemic toxicity and be non-allergenic. It would be effective only above some threshold of bioactivity, so that its effects could be precisely localized. It would be strong enough to sclerose the targeted tissue yet it would produce no local tissue injury if extruded into the vessel lumen 12. Examples of sclerosing materials include laureth 9 (polidocanol), morrhuate sodium, sodium tetradecyl sulfate, phenol, quinine, ethanolamine oleate, bleomycin, povidone iodine, tetracycline, doxycycline, sodium chloride (salt) and talc. Sclerosant can be manufactured in a solid pellet form or be a component of the implant.

Another class of materials suitable for this application includes neurotoxins. A neurotoxin a substance that damages, destroys, or impairs the functioning of nerve tissue. Examples include glutamate, botulinum toxin and tetanus toxin. Neurotoxin may be manufactured in a solid pellet form or be a component of the implant.

Other materials suitable for the implant 21 include polymers that cause a tissue reaction resulting in renal nerve denervation. Examples of polymers that elicit an inflammatory response resulting in tissue fibrosis and renal nerve denervation include non-cured and fully cured cyanoacrylate (2-octyl cyanoacrylate), Dacron™ fibers and meshes (polyethylene terephthalate), absorbable surgical suture materials such as polyglycolic acid, polylactic acid, and polydioxanone, non-absorbable surgical suture materials such as nylon, polyester, and polypropylene. Additional organic materials sufficient for the implant 21 include surgical suture materials silk, gut (collagen) and chromic gut.

Relatively less tissue responsive polymer materials coated or impregnated with bioactive components that cause renal denervation can also be employed. Examples of these materials include porous materials such as sintered high density polyethylene, Gortex™ (expanded polytetrafluoroethylene) and porous silicone. Any of the aforementioned sclerosants and neurotoxin materials may also be suitable as coatings and impregnations. Hydrogels, which are non-expanded solid materials ex-vivo and expand in size in-vivo may also be suitable for this application and include for example poly (hydroxyethylmathacrylate), polyacrylamides, N-vinyl-2-pyrrolidone, methacrylic acid, methyl methacrylate and maleic anhydride.

Other materials suitable for the implant 21 include metals that cause a tissue reaction resulting in renal nerve denervation. Examples of these include 316L stainless steel, cobalt based alloys (e.g. MP35N, and Elgiloy) and titanium alloys (e.g. Nitinol). The implants may have a straight or curved cylindrical type shape. Alternatively, they can have the shape adjusted after implantation. They may also have shape memory properties (such as implants composed of Nitinol) which allows for their shape to assume a predetermined shape after implantation. Use of implants made of materials which have shape memory properties permit the implant to assume a preset shape after insertion. Alternatively, certain conditions may be applied, such as application of heat, cold, light or a magnetic field that will allow the material to assume a desired fixed or modified shape after implantation. The necessary condition will depend on the intrinsic properties of the shape memory material chosen to produce the implant 21.

Implant 21 may be compressed into needle lumen 22 so that after injection, implant expands to its pre-compression dimension or shapes which assists with preventing implant migration and extrusion. Edema or tissue swelling from the implant 21 bioactivity as well as the injury to the tissue caused by the needle element 18 vessel wall insertion may also assist with holding implant 21 in position. Implant 21 shapes may vary and include spherical, cuboidal and cylindrical. With a cylindrical shape being preferred, length can range from 100 hundred microns to 4 centimeters and radius can range from 10 microns to 5 millimeters. Implant 21 may also employ barbs, protrusions, roughened surfaces and/or in-situ shape changes to assist with retention.

It may be desirable to examine implant 21 location post insertion to confirm proper placement. In certain aspects, the implant can further include a radio-opaque, echogenic material, or MRI responsive material to aid in visualization of the device under ultrasound, fluoroscopy, and/or magnetic resonance imaging. The radio-opaque or MRI visible material may be in the form of one or more markers (e.g. bands of materials that are disposed on either end of the implant).

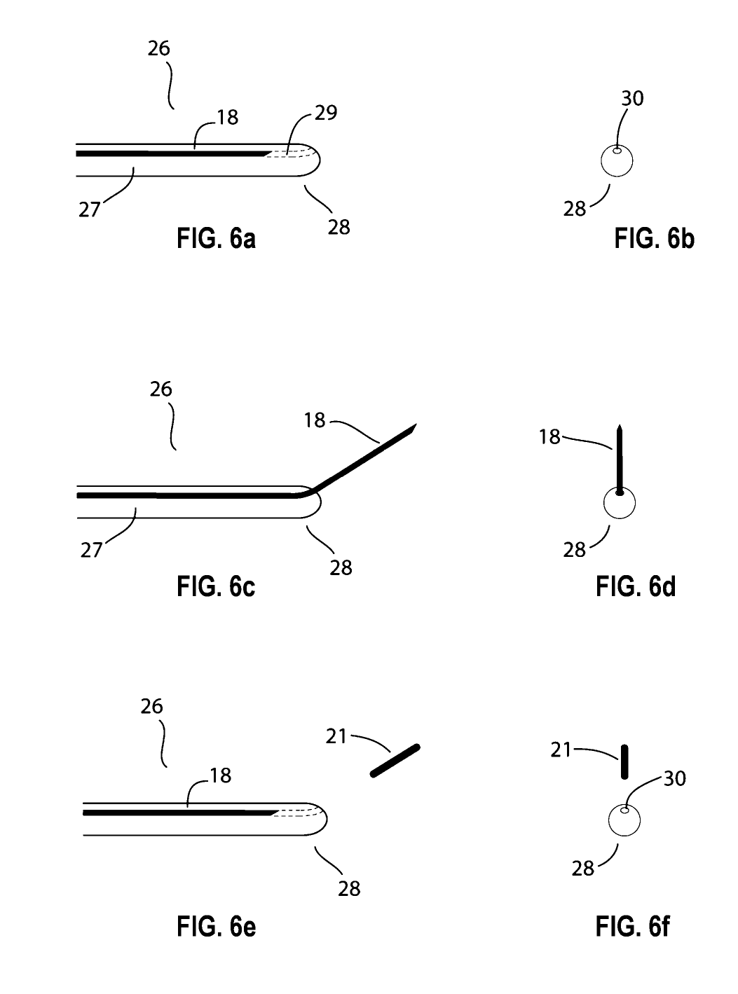

FIG. 6 is an illustration of the distal end of a delivery catheter assembly containing a single needle element used for renal denervation according to an embodiment of the present invention. In the sliced longitudinal view of FIG. 6a, a delivery catheter assembly 26 includes an elongated catheter body 27 extending longitudinally between a proximal end (not shown) and a distal end 28. Delivery catheter assembly 26 has a longitudinal length of approximately 70 centimeters with a range of 20-100 centimeters and outside diameter of approximately 0.079 inches (6 French catheter gauge) with a range of 0.039-0.131 inches (3 to 10 French catheter gauge). A needle element 18 containing implants are slidably located in a catheter lumen 29. FIG. 6b is a distal end view of the catheter assembly showing the opening 30 of catheter lumen 29 located at twelve o'clock. FIGS. 6c-6d illustrate distal advancement of needle element 18 through catheter lumen 29. Upon exit of catheter lumen opening 30, needle element 18 deflects from longitudinal axis approximately 20-70 degrees. Needle element 18 deflection may be accomplished by physical deflection of the needle element 18 through the curved channel catheter lumen 29 or needle element 18 may have a preformed curved shape that is constrained in the catheter lumen 29 in a relatively straight configuration and then allowed to form into its preformed curved angle upon exit of lumen opening 30. In other embodiments not shown, needle elements 18 can also exit from any location on the lateral, circumferential surface of the catheter body 27. Hypodermic needle 19 materials include metals such as stainless steel, shape memory alloys such as Nitinol and rigid plastics such as liquid crystal polymers, polyimides and polyetheretherketone.

FIGS. 6e-6f illustrate proximal retraction of the needle element 18 back into delivery catheter assembly 26 after placement of implant 21 into vessel wall (not shown). In the single needle element 18, delivery catheter embodiment there contains multiple implants 21 within the needle element 18. In use, delivery catheter assembly 26 is inserted into a blood vessel or the like with needle element 18 fully retracted in catheter body 27 as shown in FIGS. 6a and 6b. Once in position, needle element 18 is advanced out of the catheter body 27 and into the vessel wall where one or more implants 18 are deposited (FIGS. 6c-6d). Needle element 18 is then retracted back into the catheter body 27 (FIGS. 6e-6f) and the delivery catheter distal end 28 is maneuvered axially and/or longitudinally within the renal vessel lumen 12 for the next implant placement procedure. Implant placement and distal catheter 28 maneuvering procedures are repeated until an adequate number of implants 21 are deposited within the vessel wall to create a remodeled zone that captures a full loop of tissue perpendicular to the longitudinal axis of the vessel that results in a circumferential block of nerve impulses and renal denervation.

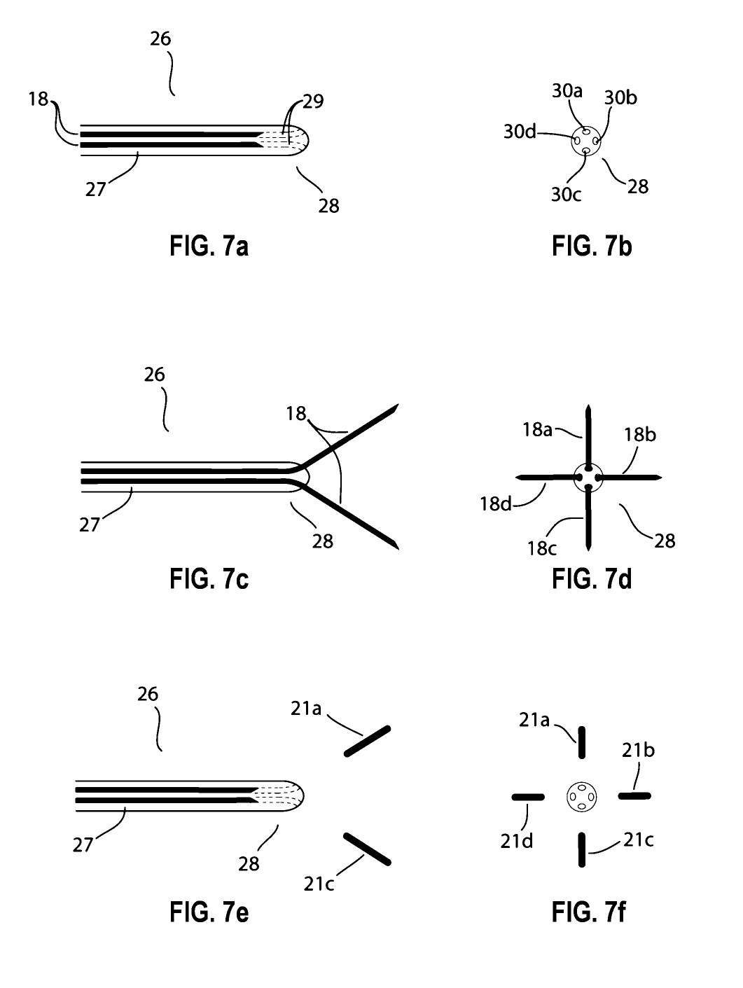

FIG. 7 is an illustration of the distal end of a delivery catheter assembly 26 similar to the delivery catheter assembly 26 of FIG. 6. They differ primarily in the quantity of needle elements 18. In FIG. 6, the needle element exits a single catheter lumen 29 shown at twelve o'clock. As shown in FIG. 7b, delivery catheter assembly 26 contains four catheter lumen openings 30, 30a shown at twelve o'clock, 30b shown at three o'clock, 30c shown and six o'clock and 30d shown at nine o'clock. Needle elements 18 are advanced and implants 21 deposited in vessel wall as previously described for delivery catheter assembly 26 of FIG. 7. Needle elements 18 can be advanced concurrently as shown in FIGS. 7c and 7d or in series (not shown). FIG. 7 illustrates one of many ways to incorporate needle elements 18 to a delivery catheter assembly 26 and is not meant to limit the possible needle element 18 configurations and quantities incorporated to said delivery catheter assembly 26.

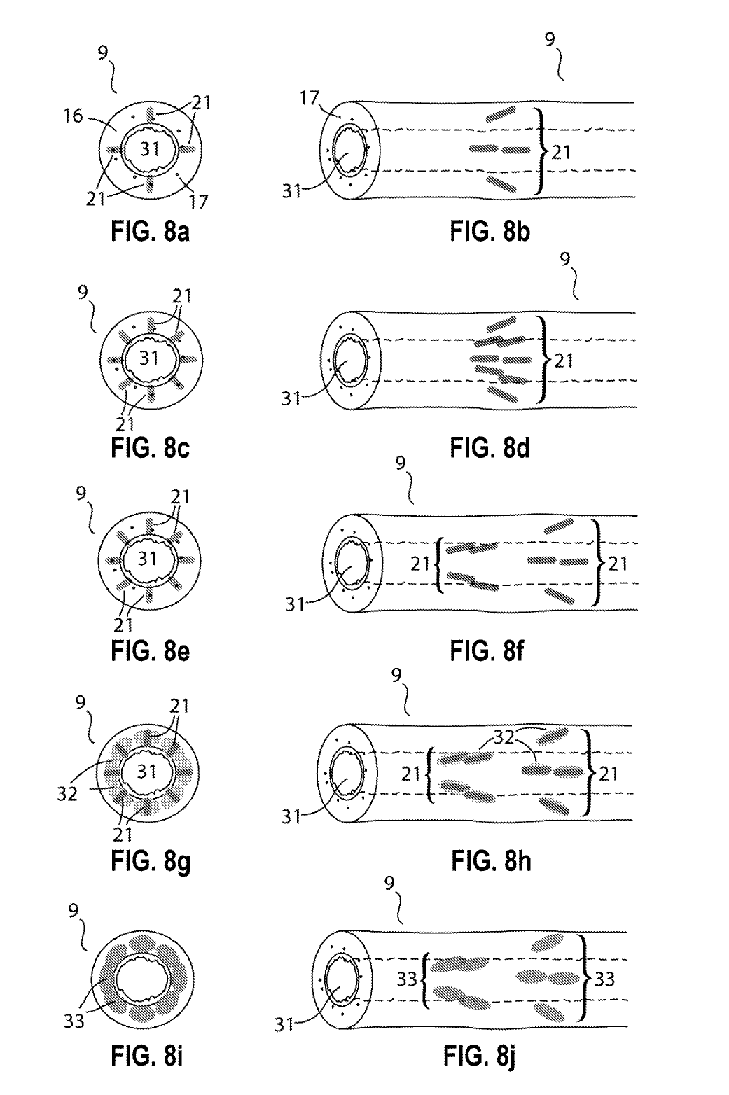

FIG. 8 is an illustration of a renal artery 9 clinically treated using the delivery catheter assembly 26 of FIG. 7. In use, the delivery catheter assembly 26 is inserted into the proximal opening 31 of renal artery for treatment. FIG. 8a is a cross-sectional view and FIG. 8b is a perspective longitudinal view of the renal artery 9 with four implants 21 deposited within the tunica adventitia 16 or vessel wall. Implants 21 are located in proximity to renal nerves 17 which run in a relatively longitudinal direction alongside the renal artery 9 within the tunica adventitia 16. In one embodiment, delivery catheter assembly 26 of FIG. 7 contains multiple implants 21 in the needle elements 18 as shown in FIG. 5. Needle elements 18 of this embodiment are positioned circumferentially within discrete segments of the catheter body 27 perpendicular to the longitudinal axis of the delivery catheter assembly 26. After first delivery of implants 21 using this embodiment, catheter distal end 28 is axially rotated approximately 45 degrees and second implant procedure is completed resulting in eight implants 21 placed circumferentially in vessel wall (FIG. 8c-8d). Alternatively, using the same embodiment, after first delivery of four implants 21, the delivery catheter distal end 28 is axially rotated approximately 45 degrees, moved longitudinally approximately one centimeter and second implant procedure is completed. Resulting in eight implants 21 placed staggered from a longitudinal view (FIG. 8f) but forming a loop circumferentially from a cross-sectional view (FIG. 8e). FIGS. 8g and 8h illustrate the initiation of a tissue response 32 surrounding the implants 21. Disruption and healing of the tissue continues and eventually leads to a permanent remodeling of the tissue, identified as the remodeled zone 33 in FIG. 8i. The remodeled zones 33 span several open arc segments around the longitudinal axis, but the zones span a substantially closed loop around the longitudinal axis as illustrated in FIG. 8i, creating an effective blockage of nerve activity.

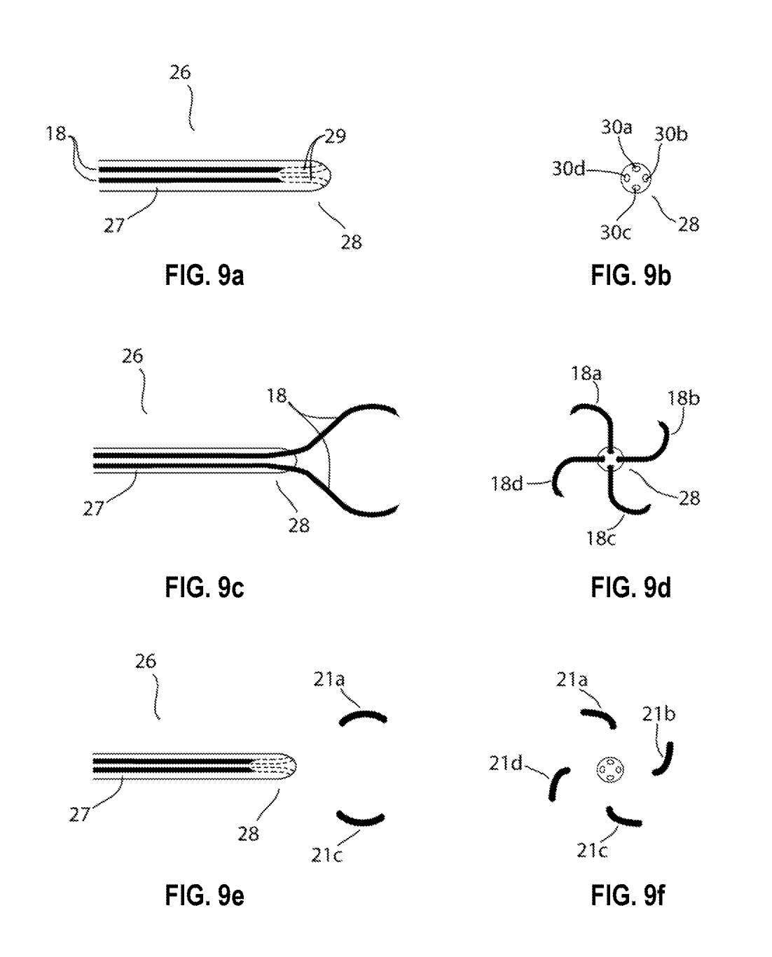

FIG. 9 is an illustration of the distal end of a delivery catheter assembly 26 similar to the delivery catheter assembly 26 of FIG. 7. Embodiments differ primarily in the spiral shape of the needle elements 18. Needle elements 18 containing implants 21 are slidably located in catheter lumens 29. FIG. 9b is a distal end view of the catheter assembly showing the openings 30 of catheter lumen 29. FIGS. 9c-9d illustrate distal advancement of needle elements 18 through catheter lumens 29. Upon exit of catheter lumen openings 30, needle elements 18 deflects from longitudinal axis to a laterally outward spiral shape. Needle elements 18 have a preformed curved shape that is constrained in the catheter lumen in a relatively straight configuration and then allowed to form into its preformed spiral shape upon exit of catheter lumens 29. Hypodermic needle 19 and/or holding rod 24 of needle element 18 may be manufactured with any of the shape memory metals such as Nitinol.

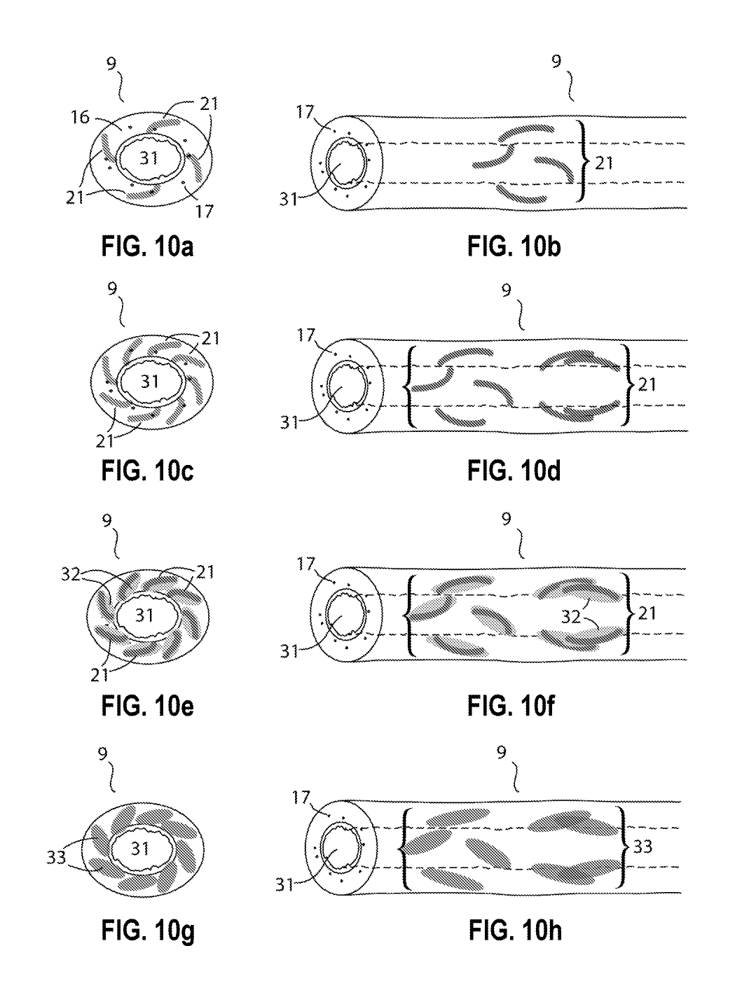

FIG. 10 is an illustration of a renal artery 9 clinically treated using the delivery catheter assembly 26 of FIG. 9. Similarly to device and method descriptions in FIGS. 7 and 8, FIG. 10 illustrates the implant 21 deposition locations within the tunica adventitia 16. Implant 21 placements lead to remodeled zones 33 that span several open arc segments around the longitudinal axis, but remodeled zones 33 span a substantially closed loop around the longitudinal axis as illustrated in FIG. 10h, creating an effective blockage of renal nerve activity.

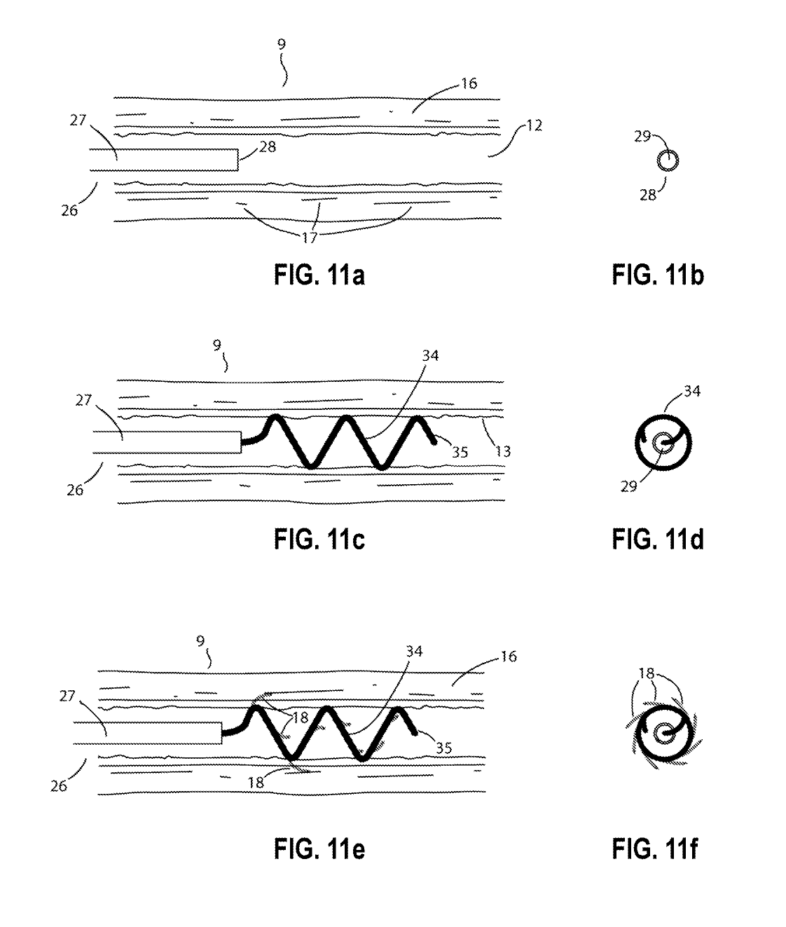

FIG. 11 is an illustration of the distal end of a delivery catheter assembly 26 comprising a coil element assembly used for renal denervation according to an embodiment of the present invention. FIG. 11a is a sliced longitudinal section of the renal artery 9 with vessel lumen 12, tunica adventitia 16 and renal nerves 17. Placed within the vessel lumen 12 is a delivery catheter assembly 26 which includes an elongated catheter body 27 extending longitudinally between a proximal end (not shown) and a distal end 28. A catheter lumen 29, as shown in the cross-sectional view of the catheter distal end 28 in FIG. 11b, houses a coil element assembly 34 in a constrained and collapsed configuration (coil element assembly not shown in FIGS. 11a and 11b). FIG. 11c illustrates the coil element assembly 34 in an expanded configuration once released from the catheter lumen 29. Coil element assembly 34 has a proximal end connected to the catheter body 27 and a distal end 35. Coil element assembly 34 expansion ceases once significant resistance occurs between coil element assembly 34 and the tunica intima 13 or inner vessel lumen surface. Coil element assembly 34 is a hollow hypo-tube with an internal diameter large enough to slidably contain needle elements 18. Needle elements are projected laterally outward through holes in the walls of the coil element assembly 34 into the vessel wall for implant 21 placement (FIGS. 11e and 11f).

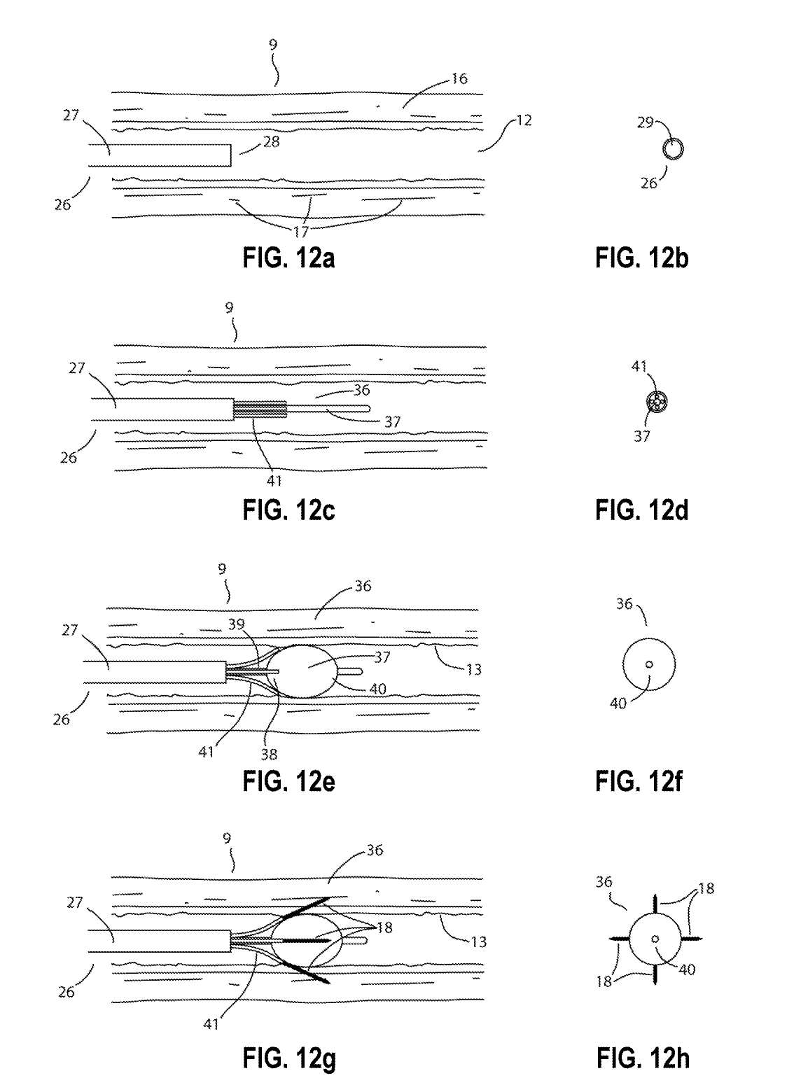

FIG. 12 is an illustration of the distal end of a delivery catheter assembly 26 comprising a balloon element assembly 36 used for renal denervation according to an embodiment of the present invention. FIG. 12a is a sliced longitudinal section of the renal artery 9 with delivery catheter assembly 26 placed within the vessel lumen 12. FIG. 12b is a distal end view of the delivery catheter assembly 26. FIGS. 12a and 12b illustrate a delivery catheter assembly 26 that includes an elongated body 27 extending longitudinally between a proximal end (not shown) and a distal end 28 with catheter lumen 29 extending the length of catheter body 27. Housed within the catheter lumen 29 is a deflated and collapsed balloon element assembly 36 (not shown in FIGS. 12a and 12b).

Balloon element assembly 36 illustrated in FIGS. 12c-12h, is similar in design to the balloons manufactured for coronary angioplasty catheters. Balloon element 37 has a proximal surface 38 connected to an inflation tube 39 and a distal surface 40. Inflation tube 39 comprises an elongated body extending longitudinally between proximal end (not shown) and a distal end within length of catheter lumen 29. Balloon element assembly 36 is movable between a collapsed configuration and an expanded configuration. As shown in FIGS. 12e-12h, one possible configuration includes an elongated spherical shaped balloon element 37 when inflated. Balloon element may be manufactured with a relatively thin walled compliant or noncompliant plastic. Examples of materials used to manufacture the balloon element 37 include polyethylene, polyethylene terephthalate, nylon and silicone elastomers. Tubular elements 41 with an elongated body extending longitudinally between a proximal end (not shown) and a distal end with an internal diameter large enough to slidably contain needle elements 18 are attached to the proximal balloon surface 38.

Balloon element assembly 36 may be inflated and deflated similarly to techniques used for angioplasty, for example by use of a pneumatic indeflator attached to the proximal end of inflation tube 39. In use, balloon element assembly 36, is advanced distally out of catheter lumen 29 and placed at targeted treatment site within vessel lumen 12 (FIG. 12c). Compliant balloon element assembly 36 is inflated and ceases expansion once significant resistance occurs between balloon element assembly 36 and the tunica intima 13 or inner vessel lumen surface (FIG. 12e). Needle elements 18 are projected laterally outward through distal end openings of the tubular elements 41 into the vessel wall for implant placement as described previously.

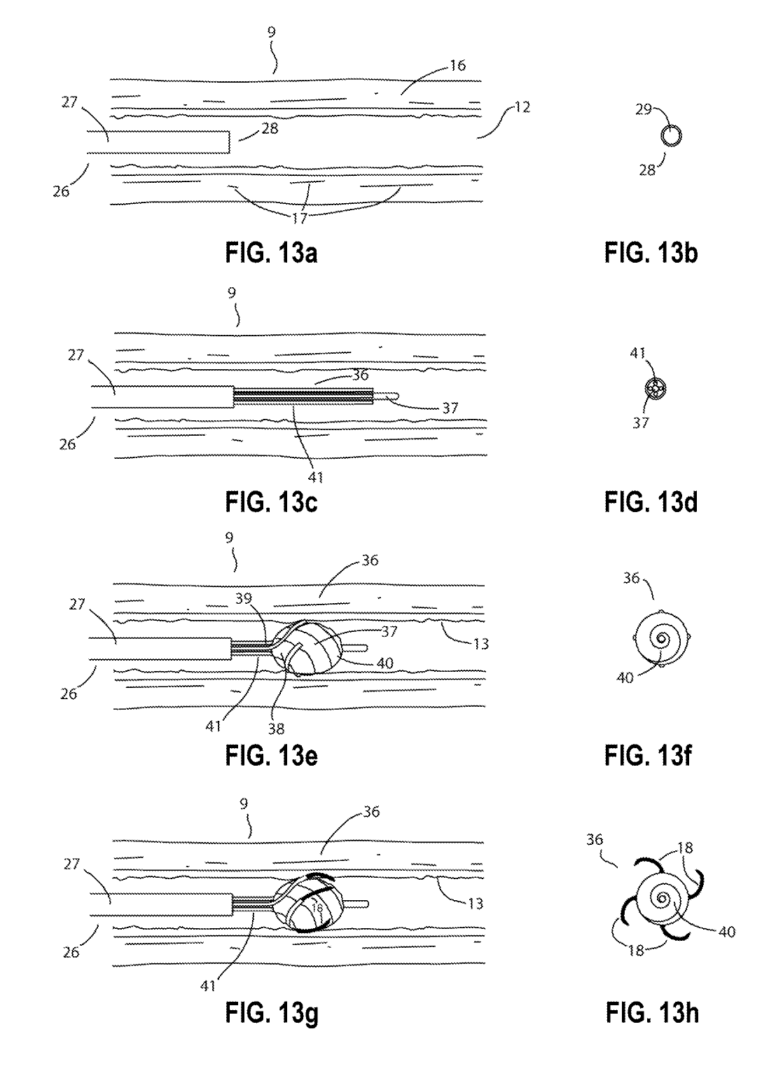

FIG. 13 is an illustration of the distal end of a delivery catheter assembly 26 similar to the delivery catheter assembly 26 of FIG. 12. Embodiments differ primarily in the spiral shape of the balloon element 37 and needle elements 18. As illustrated in FIG. 13c, balloon element 37 is a noncompliant balloon that is twisted in its deflated and collapsed configuration and tubular elements 41 slidably containing needle elements 18 are attached to the proximal balloon surface 38 in a non-twisted, straight configuration. As illustrated in FIG. 13e, balloon element 37 unfurls into an elongated spherical shape upon inflation and expansion. Unfurling action of the balloon element 37 causes tubular elements 41 to form into a laterally outward spiral configuration. FIGS. 13g and 38h illustrate distal advancement of needle elements 18 through tubular elements 41. Upon exit of distal end openings of the tubular elements 41, needle elements 18 deflects to a laterally outward spiral shape.

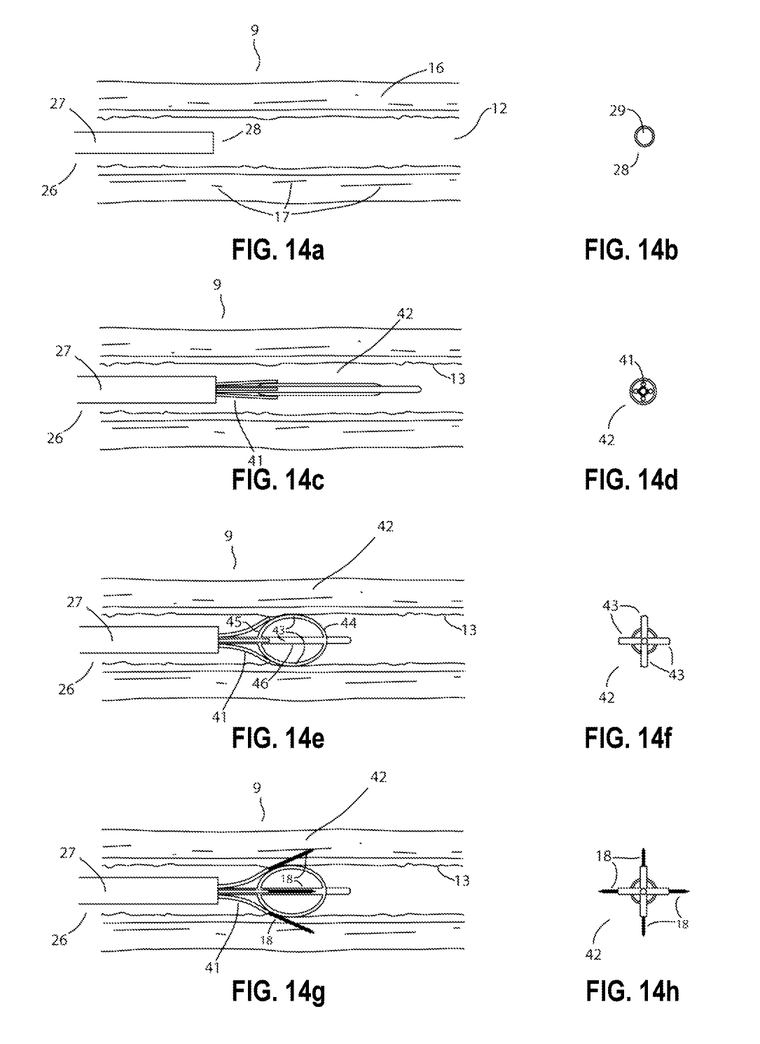

A similar device to the delivery catheter assembly 26 of FIG. 12 is illustrated in FIG. 14. Balloon assembly element 36 is replaced with a malecot element assembly 42. Malecot element assembly includes a plurality of wings 43 (four shown in illustration) which may be oriented generally longitudinally. Each wing 43 has a distal end 43 and a proximal end 45 and an intermediate segment 46. Tubular elements 41 with an elongated body extending longitudinally between a proximal end (not shown) and a distal end with an internal diameter large enough to slidably contain needle elements 18 are attached to the wing proximal end 45.

Malecot element assembly 42 is movable between a collapsed arrangement (FIGS. 14c and 14d) and an expanded arrangement (FIGS. 14e and 14f) with the intermediate segments 46 of the wings 43 in the expanded arrangement moving laterally outward relative to the distal ends 44 and proximal ends 45 of the wings 43 with respect to the collapsed arrangement of FIGS. 14c and 14d. Malecot element assembly 42 can be expanded on collapsed by various means. One example involves manufacturing wings with a memory metallic alloy (e.g. Nitinol) which have a preformed expanded shape that is constrained in the catheter lumen 29 and then allowed to recover to preformed shape upon exit of the catheter lumen 29. Another example involves mechanical expansion employing pull wire. Pull wire (not shown) is an elongated body extending longitudinally between a proximal end and a distal end, slidably contained within catheter lumen 29. Distal end of pull wire is attached to distal wing ends 44 and proximal wing ends 45 are fixed to the catheter body 27. Malecot element assembly 42 expansion occurs when pull wire is moved in a proximal and longitudinal direction relative to the catheter body 27 causing proximal wing ends 45 and distal wing ends 44 to move closer together resulting in laterally outward expansion of intermediate wings 46.

In use, the delivery catheter assembly 26 is inserted into a blood vessel in the collapsed arrangement (FIG. 14c). Malecot element assembly 42 is expanded and ceases expansion once significant resistance occurs between intermediate wings 46 and the tunica intima 13 or inner vessel lumen surface (FIG. 14e). Needle elements 18 are projected laterally outward through distal end openings of the tubular elements 41 into the vessel wall for implant placement (FIG. 14g) as previously described.

Having described this invention and methods with regards to specific embodiments, it is to be understood that the description is not meant as a limitation since further modifications may suggest themselves or be apparent to those skilled in the art. For example, variations to the above descriptions including the quantity of needle, balloon, coil and malecot assemblies and their relative positioning on the delivery catheter assembly can be easily incorporated. The application is intended to cover all such modifications and variations.

What is claimed is:

1. A method for renal denervation comprising creating a remodeled zone that spans a complete closed loop around a vessel carrying renal nerves.

2. The method of claim 1 wherein creating a remodeled zone comprises delivering a plurality of bioactive implants in proximity to renal nerves.

3. The method of claim 2 wherein delivering a plurality of bioactive implants in proximity to renal nerves comprises injecting a plurality of implants into tissue in proximity to renal nerves.

4. The method of claim 3 wherein injecting a plurality of implants into tissue in proximity to renal nerves comprises:
navigating a catheter to a target site, said catheter carrying at least one flexible needle containing at least one of said implants;
advancing said at least one needle from said catheter into said tissue;
ejecting said implants from said at least one needle.

5. The method of claim 4 wherein ejecting said implants from said at least one needle comprises retracting said needle while maintaining a position of said implants relative to said tissue.

6. The method of claim 4 wherein advancing said at least one needle from said catheter into said tissue comprises advancing said at least one needle from said catheter at an outward angle from a longitudinal axis of said catheter.

7. The method of claim 2 wherein delivering a plurality of bioactive implants comprises delivering a plurality of bioactive implants, each of said implants including a material selected from the group consisting of sclerosants, neurotoxins, polymers, and metals.

8. A device for use in renal denervation comprising:
a catheter having a plurality of lumens;
a needle contained within each lumen and moveable longitudinally relative to said catheter, each needle defining a needle lumen; and
a holding rod slideably disposed within each needle lumen;
wherein each of said needles may be retracted relative to said holding rods such that one or more implants contained in a distal end of said needle lumen is ejected from said needle lumen by said holding rod when said needle is retracted.

9. The device of claim 8 wherein each of said plurality of lumens exits a distal end of said catheter at an angle to a longitudinal axis of said catheter such that when said needles are extended from said distal end, said needles radiate outwardly from said catheter.

10. The device of claim 8 wherein said catheter comprises four lumens radially spaced ninety degrees apart from adjacent lumens.

11. The device of claim 9 wherein each of said needles comprise a preformed curve such that, upon exiting said lumens, each of said needles curves outwardly.

12. The device of claim 8 wherein each of said plurality of lumens is parallel to a longitudinal axis of said catheter and wherein each of said needles comprise a preformed curve such that, upon exiting said lumens, each of said needles curves outwardly from said longitudinal axis.

13. The device of claim 8 wherein each of said needles comprise a preformed spiral shape that is straightened when said needles are contained within said plurality of lumens but resumed when said needles exit distal ends of said lumens.

14. A device for use in renal denervation comprising:
a catheter defining a catheter lumen;
a plurality of needles slidably disposed within said catheter lumen;
at least one implant contained in a distal end of each of said plurality of needles;
a radiating mechanism for causing said needle to extend at an angle away from a longitudinal axis of said catheter when said needle is deploying said implant.

15. The device of claim 14 wherein said radiating mechanism comprises:
a coil element assembly disposed within said catheter lumen and moveable from a delivery position wherein said coil element assembly has a constrained configuration within said catheter lumen, to a deployed configuration wherein said coil element assembly extends from a distal end of said catheter lumen and expands to assume a coil shape;
wherein said coil element assembly defines a coil lumen and includes:
said plurality of needle elements slidably contained within said coil lumen;
a plurality of exit holes leading from said coil lumen, wherein each of said needle elements has a distal end that, when said needle element is advanced, extends through one of said exit holes.

16. The device of claim 14 wherein said radiating mechanism comprises exit holes defined in a sidewall of said catheter, each of said plurality of needles directed out of said exit holes when advanced.

17. The device of claim 14 wherein said radiating mechanism comprises a balloon disposed between said plurality of needles near a distal end thereof such that, upon inflation, said balloon pushes each of said needles outwardly.

18. The device of claim 14 wherein said radiating mechanism comprises preformed curves in each of said plurality of needles.

19. The device of claim 14 wherein said radiating mechanism comprises a malecot element assembly including a plurality of wings, each wing containing one of said plurality of needle elements.

20. The device of claim 19 wherein said malecot element assembly comprises nitinol.
